(19) United States
(12) Patent Application Publication
(10) Pub. No.: US 2016/0017054 A1
(43) Pub. Date: Jan. 21, 2016

(54) Spermidine/Spermine N1 - Acetyltransferase Antibodies As Anti-Cancer Drug Compounds

(71) Applicant: BIOMARK TECHNOLOGIES INC., Richmond (CA)

(72) Inventors: Brian Cheng, Chesterfield, MO (US); Rashid Bux, Vancouver (CA); Derek Cheng, Chesterfield, MO (US)

(73) Assignee: Biomark Technologies Inc., Vancouver, BC (CA)

(21) Appl. No.: 14/764,942

(22) PCT Filed: Jan. 30, 2014
(86) PCT No.: PCT/CA2014/050059

§ 371 Date: Jul. 30, 2015

Related U.S. Application Data
(60) Provisional application No. 61/758,584, filed on Jan. 30, 2013.

(51) Int. Cl. C07K 16/40

(52) U.S. Cl. CPC C07K 16/40; C07K 2317/76; C07K 2317/73

(57) ABSTRACT

An anti-cancer drug compound comprises a spermidine/spermine N¹-acetyltransferase antibody. The spermidine/spermine N¹-acetyltransferase antibody may be a monoclonal antibody or a polyclonal antibody. A method of treating cancer comprises the use of a spermidine/spermine N¹-acetyltransferase antibody. The spermidine/spermine N¹-acetyltransferase antibody may be a monoclonal antibody or a polyclonal antibody.

### BACKGROUND OF THE INVENTION

1. Field of the Invention

The present invention relates to the use of spermidine/spermine N¹-acetyltransferase (SSAT) antibodies as anti-cancer drug compounds and in anti-cancer treatments.

2. Description of the Related Art

U.S. Pat. No. 6,811,967 which issued to Sitar et al. on Nov. 4, 2004, and the full disclosure of which is incorporated herein by reference, discloses a method for assaying activity of the enzyme SSAT using SSAT substrates by detecting acetylated forms of the SSAT substrates. Disclosed also is the correlation of SSAT activity to pathological conditions.

SSAT is an important enzyme in polyamine metabolism. Polyamines, including spermidine and spermine, are essential for cell survival and SSAT is a rate-limiting enzyme in the catabolic pathway which converts spermidine and spermine into acetylpolyamines to maintain intracellular polyamine homeostasis. It has been reported that in certain cancer cell lines a high expression of SSAT mRNA have been detected. See, for example, Chen et al. Genomic identification and biochemical characterization of a second spermidine/spermine N¹-acetyltransferase. Biochemical Journal. (2003), Volume 373, 661-667, the full disclosure of which is incorporated herein by reference.

It has also been reported that SSAT expression and enzymatic activity may be elevated following chemotherapy or treatment with spermidine analogues. In vitro cell line studies have further positively correlated SSAT expression and enzymatic activity with levels of cytotoxicity of new drug candidates. A number of anti-proliferative agents and polyamine analogues have accordingly been developed to prevent cancer cell proliferation via SSAT induction. See for example, Wallace, H. M. et al. A perspective of polyamine metabolism. Biochemical Journal. (2003), Volume 376, 1-14, the full disclosure of which is incorporated herein by reference.

### SUMMARY OF THE INVENTION

It is an object of the present invention to provide improved anti-cancer agents and anti-cancer treatments.

Certain cancer cells have intracellular spermidine/spermine N¹-acetyltransferase SSAT to acetylate polyamines to balance intracellular pH and excrete extracellular. However, certain levels of intracellular polyamines may be cytotoxic. Cancer cells have higher levels of intracellular polyamines. The retention of these intracellular polyamines may cause cancer cell death. There is accordingly provided an anti-cancer drug compound which comprises a spermidine/spermine N¹-acetyltransferase antibody. There is also provided a method of anti-cancer treatment which utilizes an SSAT antibody to suppress the SSAT acetylation of polyamines thereby causing cancer cell death. The antibody may be a monoclonal or a polyclonal antibody.

### BRIEF DESCRIPTIONS OF DRAWINGS

The invention will be more readily understood from the following description of the embodiments thereof given, by way of example only, with reference to the accompanying drawings, in which:

FIG. 1 shows the relative SSAT expression levels by RT-qPCR assay and metabolic activities as measured by N-acetylspermidine formation in U2-OS, HeLa, Malme-3M, PC-3 and HEK293 human tumor cell lines;

FIG. 2 also shows the relative SSAT expression levels by RT-qPCR assay and metabolic activities as measured by N-acetylspermidine formation in U2-OS, HeLa, Malme-3M, PC-3 and HEK293 human tumor cell lines;

FIG. 3 shows the relative percent confluency of human tumor cell lines, U2-OS, HeLa, Malme-3M, PC-3 and HEK293 during incubation with spermidine from 22 μM to 550 μM;

FIG. 4 shows a cytotoxicity of a SSAT polyclonal antibody and spermidine as the polyamine positive control against the lung epithelial cancer cell line A549;

FIG. 5 shows a cytotoxicity of a SSAT polyclonal antibody and spermidine as the polyamine positive control against the prostate adenocarcinoma cell line LNCaP;

FIG. 6 shows a cytotoxicity of a SSAT polyclonal antibody and spermidine as the polyamine positive control against the breast epithelial cancer cell line T-47D;

FIG. 7 shows a cytotoxicity of a SSAT polyclonal antibody and spermidine as the polyamine positive control against the osteosarcoma cell line U2-OS;

FIG. 8 shows a cytotoxicity of a SSAT monoclonal antibody and the polyamine positive control spermidine against the lung epithelial cancer cell line A549;

FIG. 9 shows a cytotoxicity of a SSAT monoclonal antibody and the polyamine positive control spermidine against the prostate adenocarcinoma cell line LNCaP;

FIG. 10 shows a cytotoxicity of a SSAT monoclonal antibody and the polyamine positive control spermidine against the breast epithelial cancer cell line T-47D;

FIG. 11 shows a cytotoxicity of a SSAT monoclonal antibody and the polyamine positive control spermidine against the osteosarcoma cell line U2-OS;

FIG. 12 shows a summary of cytotoxicity of a SSAT polyclonal antibody and SSAT expression levels in human cell lines;

FIG. 13 shows a summary of cytotoxicity of a SSAT monoclonal antibody and SSAT expression levels in human cell lines;

FIG. 14 shows a cytotoxicity of results of monoclonal and polyclonal SSAT antibodies against various tumor cell lines;

FIG. 15 shows a correlation of SSAT expression in A549, LNCaP and T-47D cell lines against antitumor potency (IC₅₀) of SSAT antibody;

FIG. 16 shows SSAT expression levels in human cancer cell lines relative to A549 using GAPDH or hPRT1 house-keeping genes as an internal reference; and

FIG. 17 also shows SSAT expression levels in human cancer cell lines relative to A549 using GAPDH or hPRT1 house-keeping genes as an internal reference.

### DESCRIPTIONS OF THE PREFERRED EMBODIMENTS

A method of using spermidine/spermine N¹-acetyltransferase (SSAT) antibodies as anti-cancer drug compounds is disclosed herein.

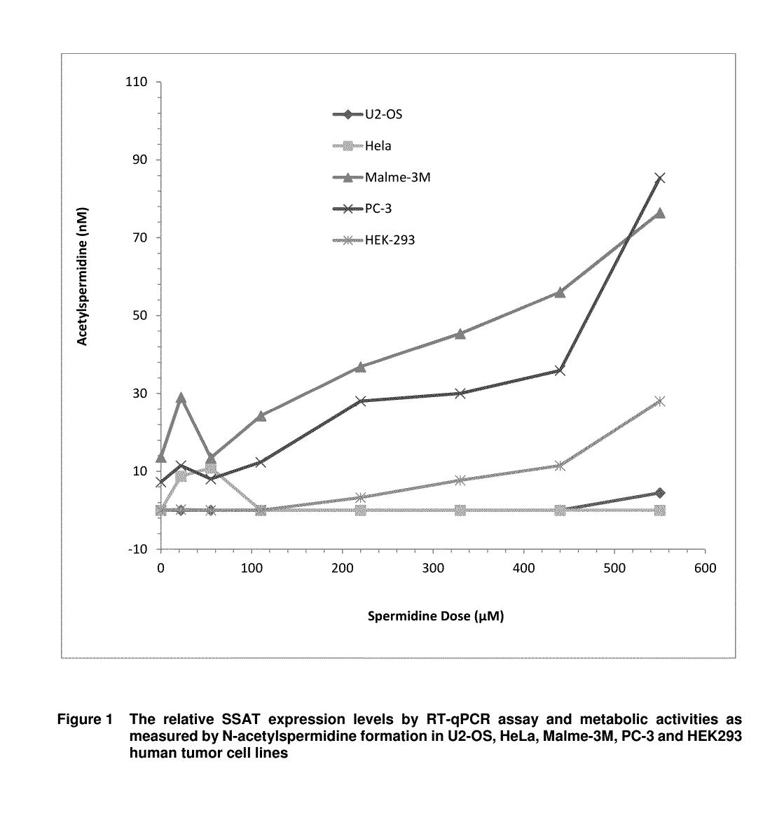

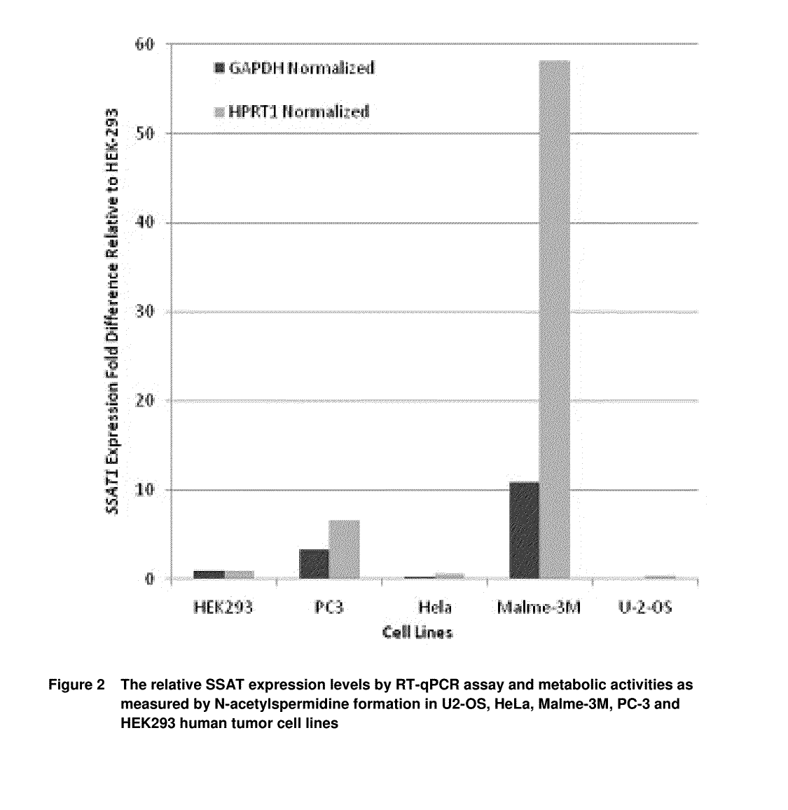

The relative SSAT expression levels in human tumor cell lines, HEK-293, Malme-3M, HeLa, PC-3 and U2-OS cell lines were determined by a reverse transcription—qualitative polymerase chain reaction assay (RT-qPCR assay) and, as shown in FIGS. 1 and 2, Malme-3M was observed with the highest relative SSAT expression at 11-fold more than that of the control HEK-293 cell line when normalized with GAPDH, and 58-fold more when normalized with HPRT1. PC-3 had the second highest expression level with approximately 3-fold and 7-fold differences of SSAT expression relative to HEK-293 when normalized with GAPDH and HPRT1, respectively. Both HeLa and U2-OS had lower SSAT expression levels than HEK-293. The SSAT expression levels were also compared against N-acetylated amantadine metabolite formation and the findings suggested a causal relationship between SSAT expression and N-acetylation metabolic activity.

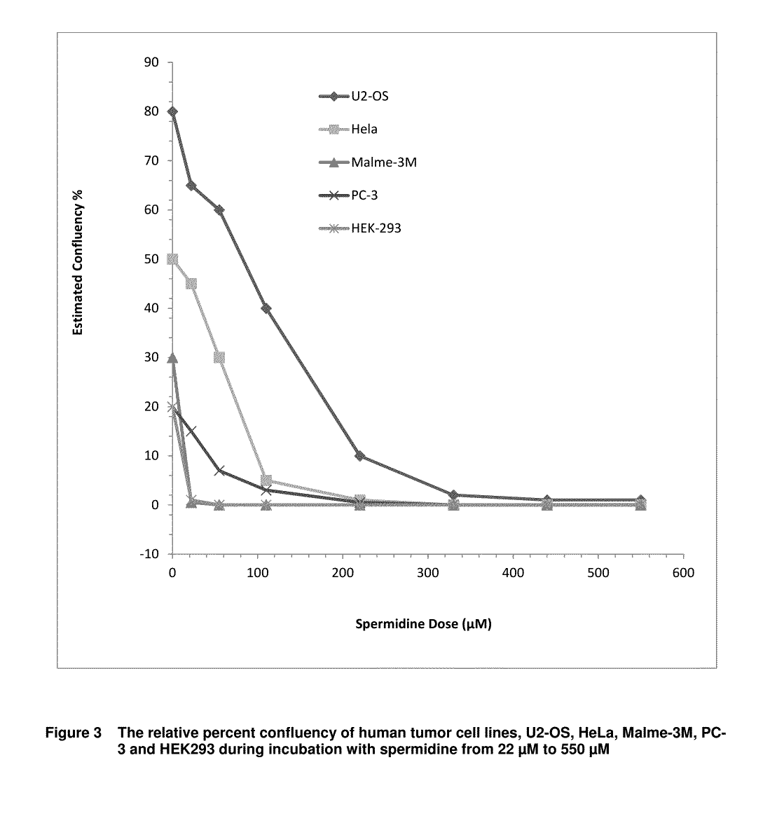

Referring now to FIG. 3, when the human tumor cell lines were incubated in the presence of spermidine from 22 μM to 550 μM, the relative cell viability, expressed as percent confluency, was observed to be highest in SSAT non-expressing cell lines (U2-OS and HeLa) with the lowest SSAT N-acetylation activity. In contrast, cell viability was observed to be lowest in SSAT over-expressing cell lines (Malme-M3 and PC-3). This data suggests that the significantly high cytotoxicity of spermidine in human tumor cell lines is mediated by a metabolism-based mechanism of SSAT in tumor cells over-expressing SSAT.

### Materials

### Antibodies

The following antibodies were evaluated for cytotoxicity against human cancer cell lines.

| Identity: | SSAT Antibody |
|---|---|
| Clonality/Epitope: | Rabbit Polyclonal, C-terminal residues, N-terminal |
| | residues |
| Purity: | Peptide affinity purified |
| Supplier: | Novus Biologicals, LLC |
| Clonality/Epitope: | Mouse Monoclonal |
| Batch/Lot Number: | A003 |
| Expiry Date: | Sep. 14, 2013 |
| Purity: | Peptide affinity purified |
| Supplier: | OriGene Technologies Inc. |

### Positive Control

Spermidine being a polyamine substrate for SSAT was used as a positive control test drug in this study.

| Identity: | Spermidine |
|---|---|
| BRI Reference No: | RFS-1085 |
| Purity: | 99.8% |
| Batch/Lot No.: | 1441607 |
| Supplier: | Sigma Pharmaceuticals, LLC |

### Vehicle Controls

The following vehicle control was used for the monoclonal antibody in this study.

| Identity: | Sterile Phosphate Buffered Saline (PBS) at pH 7.4 |
|---|---|
| BRI Reference No: | STM-1583 |
| Batch/Lot No.: | SS-7011-1 |
| Expiry Date: | Jan. 27, 2017 |
| Supplier: | Sigma Pharmaceuticals, LLC |

The following vehicle control was used for the polyclonal antibody in this study.

| Identity: | Sterile Phosphate Buffered Saline (PBS) at pH 7.4 |
|---|---|
| Batch/Lot No.: | SS-7011-1 |
| Supplier: | Prepared in-house |

The following vehicle control was used for the monoclonal antibody in this study.

| Identity: | Sterile Phosphate Buffered Saline (PBS) at pH 7.4 |
|---|---|
| BRI Reference No: | STM-1583 |
| Batch/Lot No.: | SS-7011-1 |
| Expiry Date: | Jan. 27, 2017 |
| Supplier: | Sigma Pharmaceuticals, LLC |

Human Cell Lines

The following cancer cell lines were used.

| Cell Line Designation | ATCC¹ Number | Cancer Type |
|---|---|---|
| A549 | CCL-185 | Lung Epithelial Cancer |
| LNCaP | CRL-1740 | Prostate Adenocarcinoma |
| T-47D | HTB-133 | Breast Epithelial Cancer |
| U2-OS | HTB-96 | Osteosarcoma |

¹American Type Culture Collection

The evaluated cancer cell lines, namely, A549, LNCaP and T-47D are respectively human lung, prostate and breast cancer cells lines with SSAT over-expressing cancer cells. U2-OS, which is a human bone cancer cell line, was used as a SSAT non-over-expressing control.

### Experimental Procedures

Each human cancer cell line was incubated with the SSAT antibody at a range of testing concentrations. Cytotoxicity (expressed as half maximal inhibitory concentration or IC₅₀) was determined based on a MTT assay.

### Preparation of Human Cell Lines

Cells of the four cell lines were harvested from their established adherent cultures with trypsin EDTA, pelleted by centrifugation, and resuspended in the appropriate medium to yield a suspension of cells for each cell line.

### Preparation of Antibody and Positive Control Solutions

The SSAT antibody was diluted with sterile PBS into a series of antibody solutions for dosing the cell cultures. The positive control (spermidine) was accurately weighed, dissolved, and further diluted in sterile PBS into a series of spermidine solutions for dosing the cell cultures.

### Incubation With Antibody

Incubation was performed in triplicate at each antibody concentration for each cell line tested. In parallel to each antibody treatment, each cell line was also treated with spermidine as positive controls.

An aliquot of the cell suspension was added into each well of a 96-well culture plate and the plates were incubated overnight at 37° C. with a highly humidified atmosphere of 95% air and 5% carbon. On the following day each well was replaced with fresh medium and an aliquot of the appropriate antibody or positive control solution was added to achieve the following target testing concentrations:
SSAT Antibody: 0.000754, 0.00226, 0.00679, 0.0204, 0.0407, 0.0814, 0.163, 0.325, 0.813, 1.63 and 3.25 μM
Spermidine: 0.5, 1.5, 4.5, 7.5, 15, 30, 60, 200 and 600 μM

A blank vehicle was used in lieu of the substrate solutions to prepare the vehicle controls. The plates were then returned to incubation for three days at 37° C. with a highly humidified atmosphere of 95% air and 5% carbon.

### MTT Cytotoxicity Assay

An aliquot of 5 mg/mL MTT was added to each well of the three days of incubation and plates were then incubated for a further one to three hours. Following incubation the medium was replaced with DMSO to dissolve the formazan. An aliquot from each well was measured for absorbance at 550 nm or 555 nm on a 96-well flat bottom plate with a microplate reader and DMSO for background absorbance correction.

### Results and Discussion

Both SSAT polyclonal and monoclonal antibodies were evaluated for cytotoxicity against three representative human lung, prostate and breast cancer cell lines over-expressing SSAT (A549, LNCaP and T-47D, respectively). The cytotoxic potency of the SSAT antibodies was found to be equal or better than the polyamine spermidine positive control for the A549 and LNCaP cell lines. SSAT antibodies exhibited the highest potency against LNCaP with an IC₅₀ value of 4.7 μM, which is similar to the therapeutic range of existing anti-cancer therapeutic antibodies on the market. Anti-cancer cytotoxicity potency of the SSAT antibody was observed correlating positively with SSAT expression levels in the cell lines evaluated.

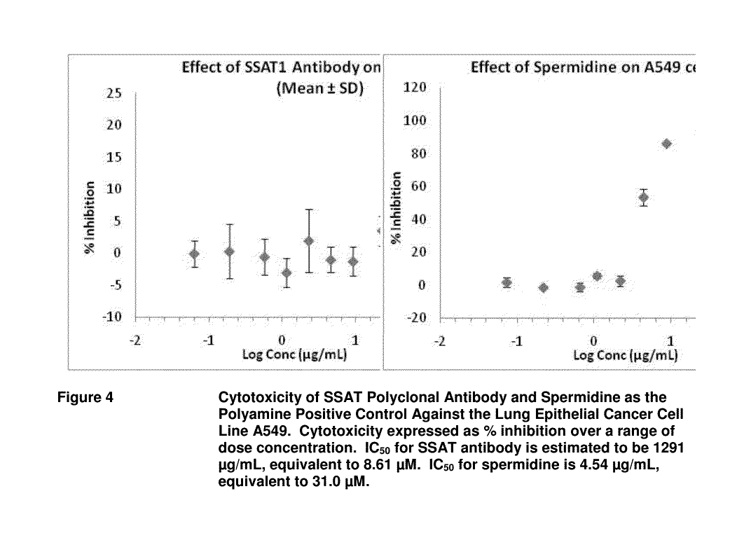

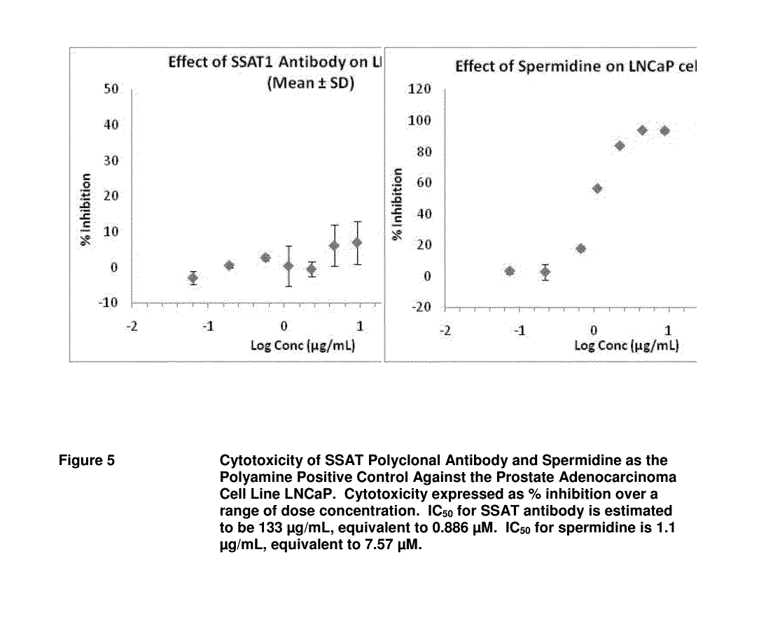

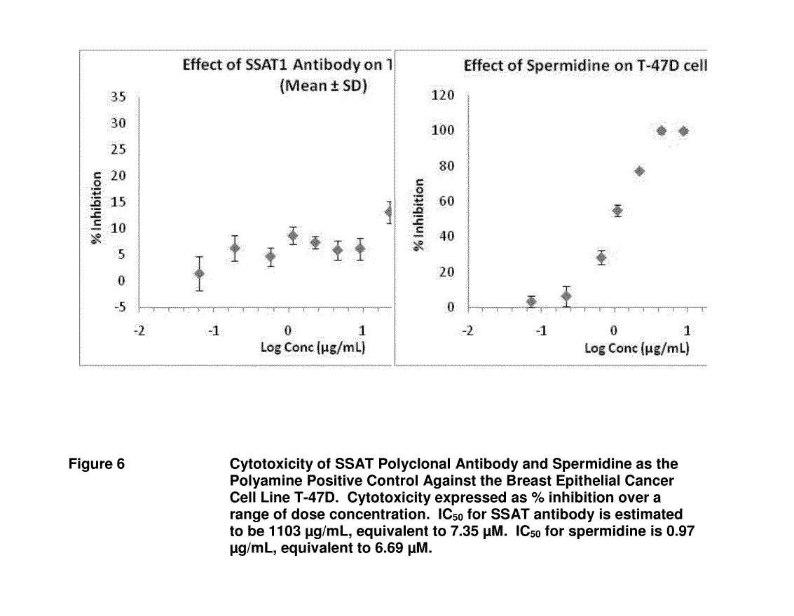

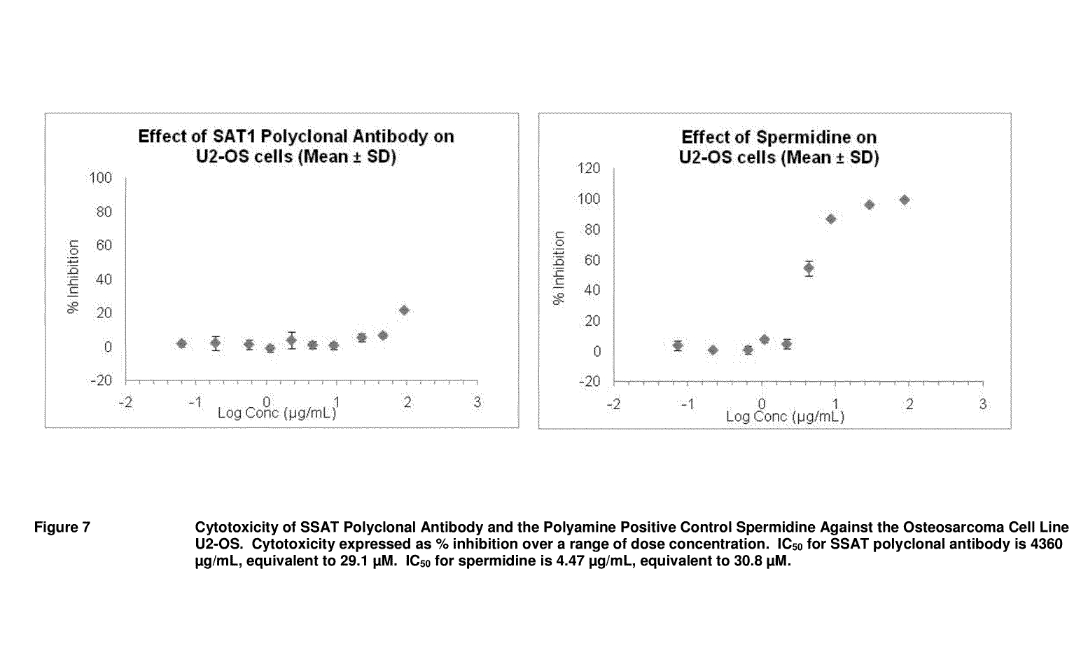

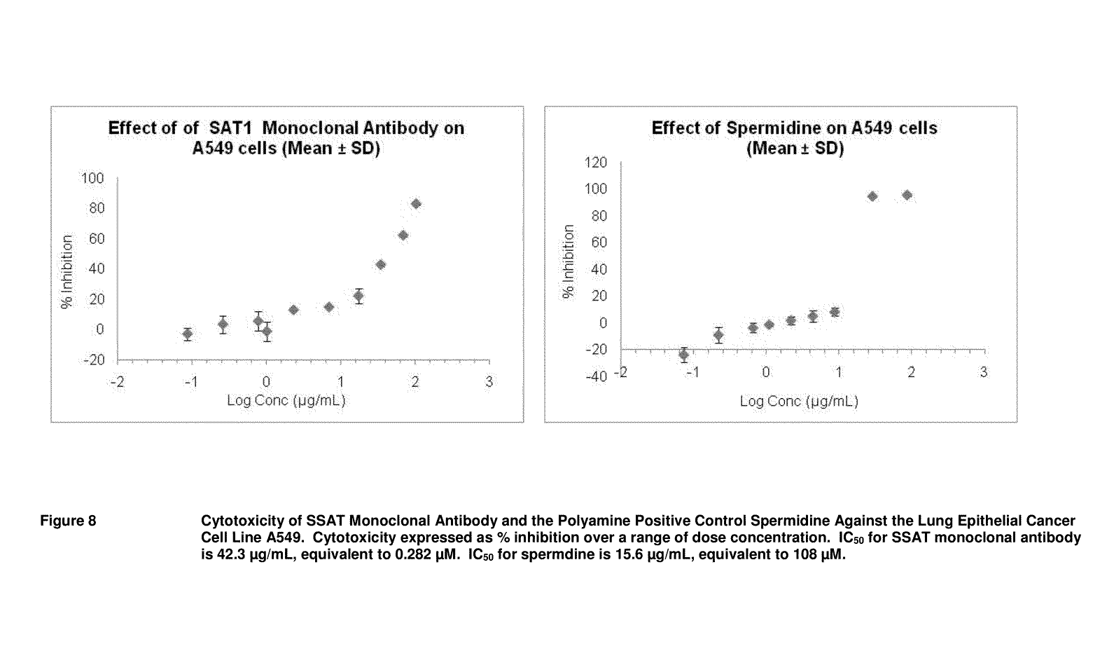

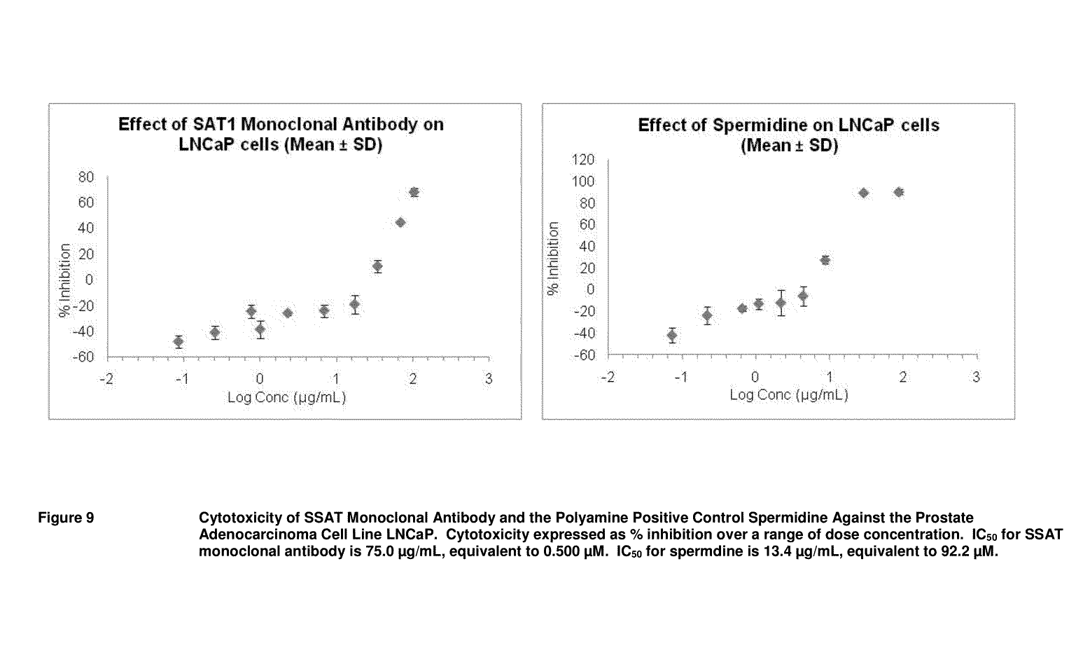

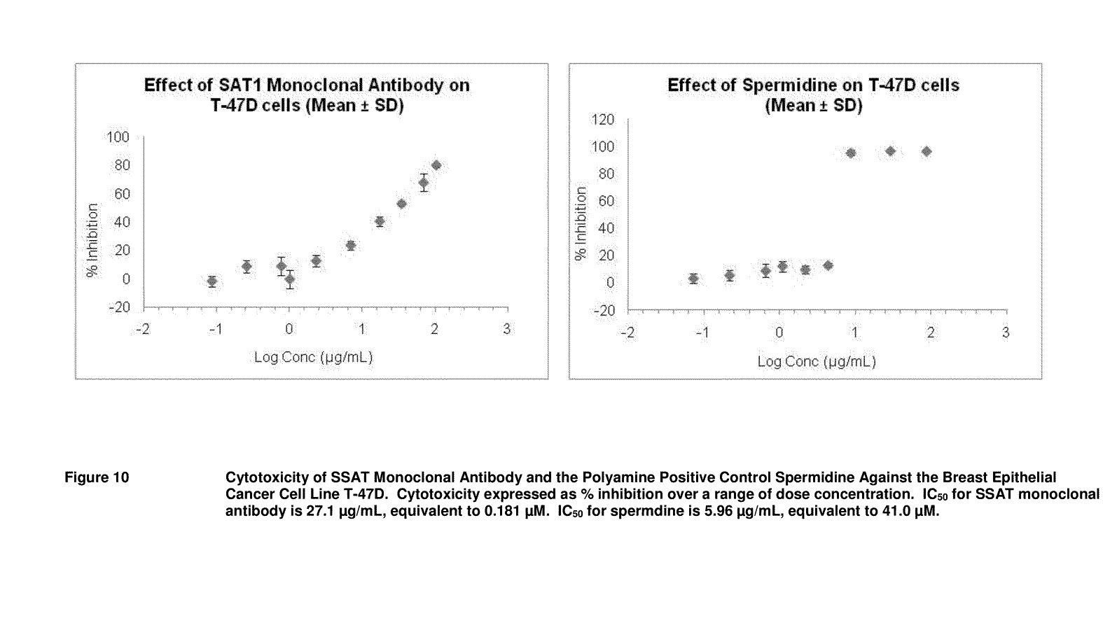

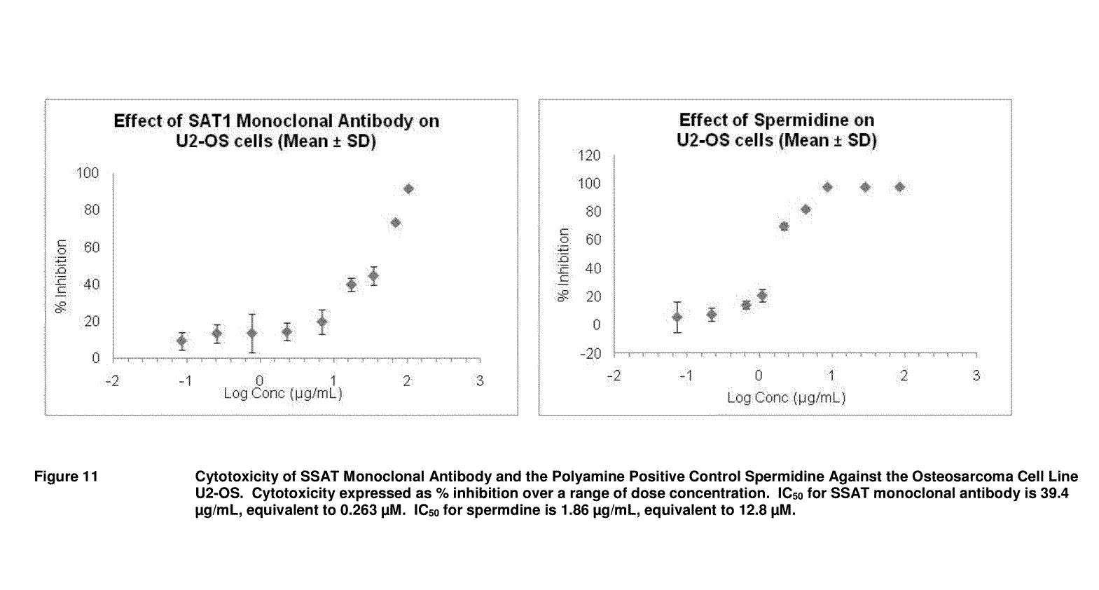

Cytotoxicity was measured by an established MTT assay following a three-day incubation with daily antibody treatment. IC₅₀ values were deduced based on plots of cytotoxicity level expressed as a percentage of inhibition over the testing concentrations as shown in FIGS. 4 to 7 for the polyclonal antibody and FIGS. 8 to 11 for the monoclonal antibody. In parallel to each drug treatment, each cell line was also treated with spermidine as positive controls and the IC₅₀ value from spermidine was determined for comparison.

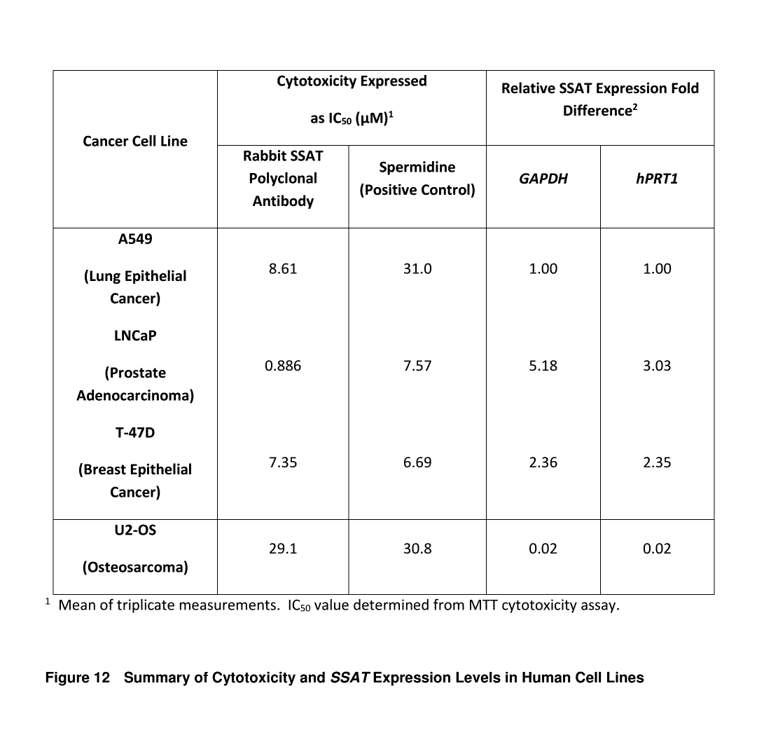

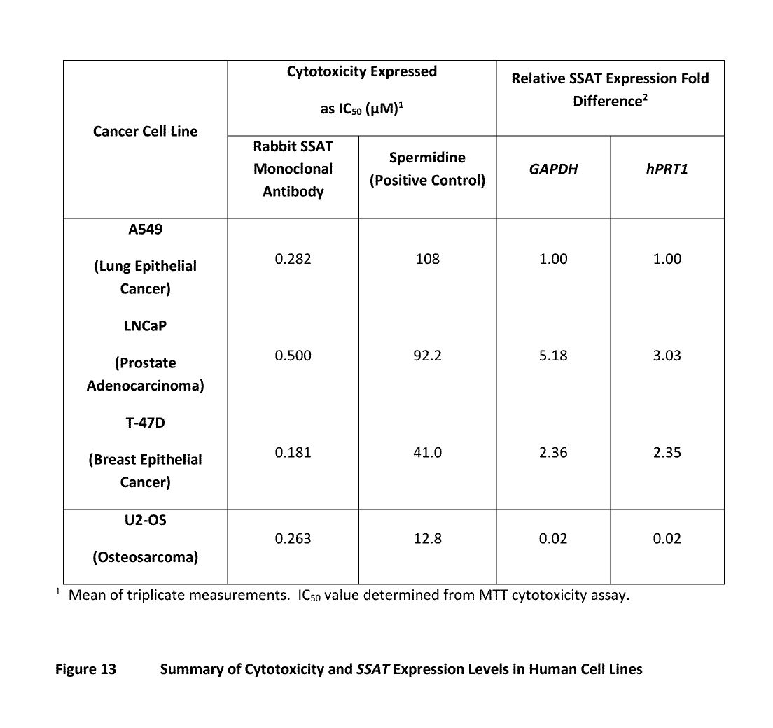

A summary of the polyclonal antibody cytotoxicity and monoclonal antibody cytotoxicity expressed as an IC₅₀ value for the cancer cell lines evaluated, namely, the A549 (lung epithelial cancer), LNCaP (prostate adenocarcinoma) and T-47D (breast epithelial cancer) human cancer cell lines is shown in FIGS. 12 and 13. Cytotoxicity IC₅₀ values were determined based on treating each cell line with the antibody over a range of concentrations. The IC₅₀ values obtained from the polyclonal antibody ranged from 0.886 μM to 8.61 μM across the three cancer cell lines evaluated. The IC₅₀ values obtained from the monoclonal antibody ranged from 0.181 μM to 0.282 μM across the three cancer cell lines evaluated.

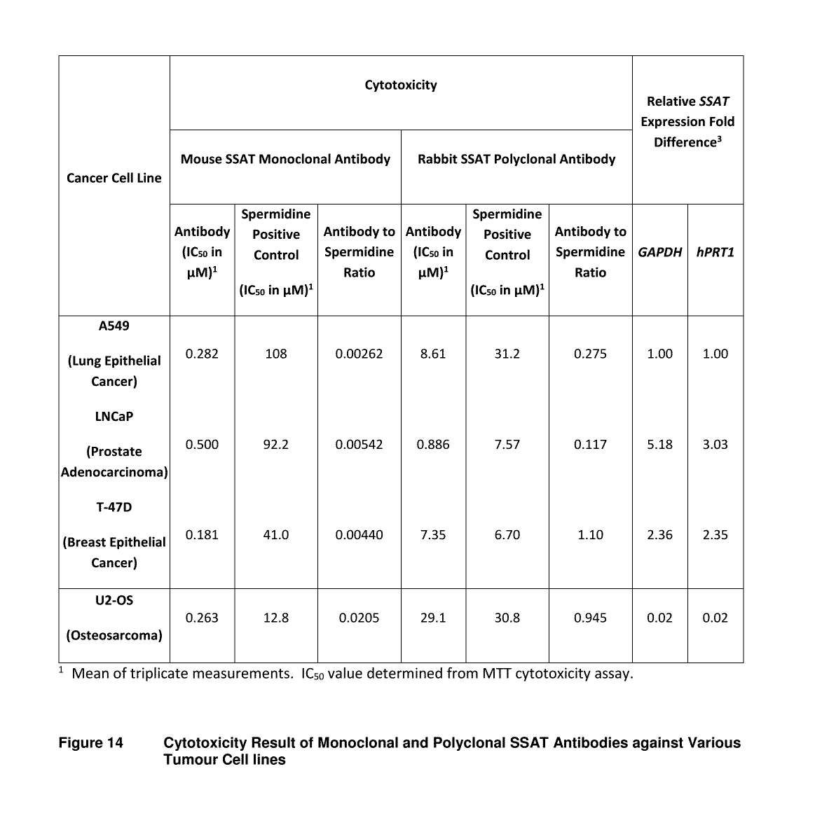

The monoclonal antibody/spermidine IC₅₀ ratios of the monoclonal antibody were compared against those obtained from the polyclonal antibody as shown in FIG. 14. The cytotoxic potency of the monoclonal antibody was found to be significantly higher across all cell lines evaluated in comparison to the polyamine spermidine positive control and the polyclonal antibody. The monoclonal antibody also had a higher efficacy against SSAT over-expressing cell lines. The ratios from the monoclonal antibody (0.00262 to 0.0205) were substantially lower than those from the polyclonal antibody (0.117 to 1.10). This suggests that the monoclonal antibody may be more potent against the cancer cell lines evaluated.

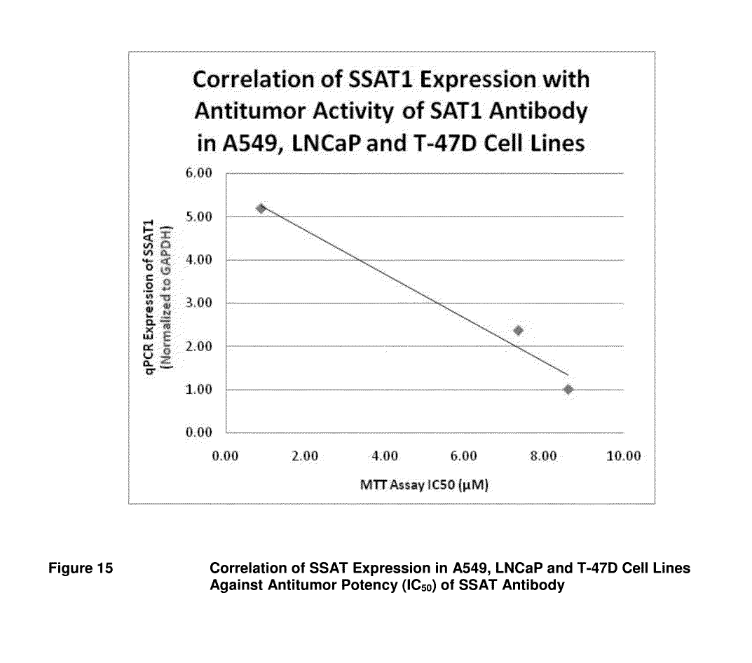

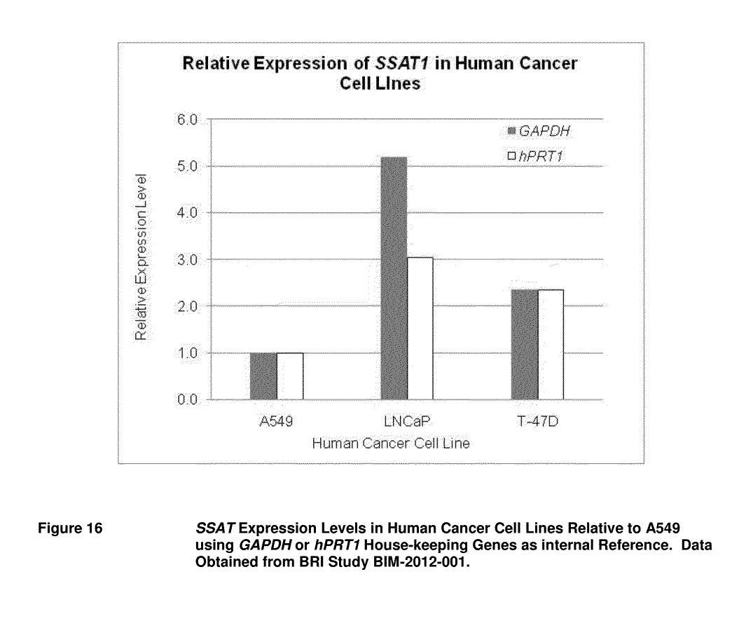

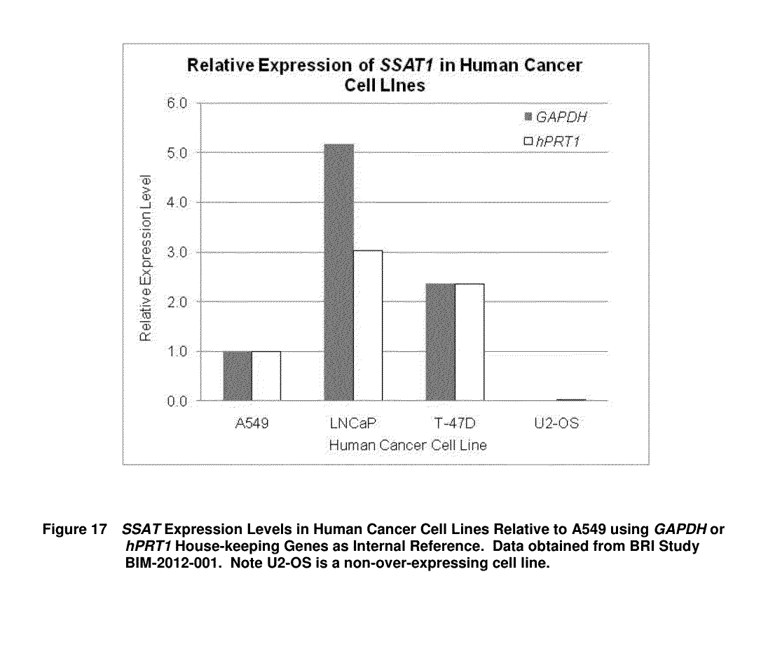

FIG. 15 shows a comparison of IC₅₀ values with the SSAT expression levels of the cell lines shown in FIGS. 16 and 17 which were obtained from a RT-qPCR assay. Cytotoxicity appears to positively correlate with SSAT expression levels. The IC₅₀ values for cancer cells are higher in SSAT over-expressing cancer cells as compared to SSAT non-over-expressing cancer cells. It is accordingly concluded that SSAT antibodies may be used as anti-cancer drug compounds and in anti-cancer treatments which target SSAT over-expressing cancer cells resulting in the retention of intracellular polyamines and cancer cell death.

It will be understood by a person skilled in the art that many of the details provided above are by way of example only, and are not intended to limit the scope of the invention which is to be determined with reference to the following claims.

1. An anti-cancer drug compound comprising a spermidine/spermine N¹-acetyltransferase antibody.

2. The anti-cancer drug compound as claimed in claim 1 wherein the spermidine/spermine N¹-acetyltransferase antibody is a monoclonal antibody.

3. The anti-cancer drug compound as claimed in claim 1 wherein the spermidine/spermine N¹-acetyltransferase antibody is a polyclonal antibody.

4. Use of a spermidine/spermine N¹-acetyltransferase antibody to treat cancer.

5. Use of a spermidine/spermine N¹-acetyltransferase antibody as claimed in claim 4 wherein the spermidine/spermine N¹-acetyltransferase antibody is a monoclonal antibody.

6. Use of a spermidine/spermine N¹-acetyltransferase antibody as claimed in claim 4 wherein the spermidine/spermine N¹-acetyltransferase antibody is a polyclonal antibody.
